(19) United States
(12) Patent Application Publication
(10) Pub. No.: US 2006/0135888 A1
(43) Pub. Date: Jun. 22, 2006

(54) Device for determining a value that is representative of accelerations as well as an ergometer

(75) Inventors: Gillian Antoinette Mimnagh-Kelleher, Eindhoven (NL); Henricus Antonius Verspaget, Eindhoven (NL); Joannes Gregorius Bremer, Eindhoven (NL); Adrianus Petrus Johanna Maria Rommers, Eindhoven (NL); Wilhelmus Lambertus Marinus Cornelius Verhoeven, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 10/544,202

(22) PCT Filed: Jan. 28, 2004
(86) PCT No.: PCT/IB04/50056

§ 371 Date: Aug. 2, 2005

(30) Foreign Application Priority Data
Feb. 7, 2003 (EP) 03100256.1

(51) Int. Cl. A61B 5/103; A61B 5/117

(52) U.S. Cl. 600/595; 128/921

(57) ABSTRACT

Device and ergometer for determining a value that is representative of the accelerations in at least two directions extending perpendicularly to each other. The device provided with a sensor system in which the acceleration in each of the directions extending perpendicularly to each other can be converted into an electric signal. The value that is representative of the accelerations can be determined from the electric signals by the signal processing means. Prior to the signal processing means, the electric signals are added together by means of an adding element.

The invention relates to a device for determining a value that is representative of accelerations in at least two directions perpendicular to each other, the device comprising a sensor system with which the acceleration in each of the mutually perpendicular directions can be converted into an electric signal while the value can be determined from the electric signals by signal processing means.

The invention further relates to an ergometer for measuring a value that is representative of a physical effort of an individual, which ergometer comprises an arrangement that includes a sensor system with which the acceleration in each of the mutually perpendicular directions can be converted into electric signals while the value can be determined from the electric signals by signal processing means.

Such a device known from US-B1-6,361,508 comprises a sensor that is suitable for measuring values that are representative of accelerations in three mutually perpendicular directions of a moving individual. The sensor generates three electric signals which are linearly proportional to the accelerations in the individual directions. The device further comprises signal processing means which include three circuits. The three separate electric signals are individually processed by the three circuits.

A disadvantage of this device is that the device is relatively complex and expensive as a result of the three circuits.

It is an object of the present invention to provide a device with a relatively simple and cost-effective structure by means of which a value that is representative of the accelerations can be obtained.

With the device according to the invention this object is achieved in that the electric signals can be added together by means of an adding element preceding the signal processing means.

By adding the electric signals together prior to the signal processing means, only a single electric circuit then needs to be used for determining the value. This provides a device with a simple structure that can furthermore be manufactured in a compact and cost-effective way.

An embodiment of the device according to the invention is characterized in that in the adding element the connections conducting the electric signals are connected in parallel.

In such manner the signals are added together in a simple and compact manner by means of the connections already present.

Another embodiment of the device according to the invention is characterized in that the sensor system comprises at least a sensor that includes a flexible strip made of piezoelectric material.

Such a sensor can be manufactured cost effectively while with the aid of the sensor relatively accurate accelerations in and contrary to a direction perpendicular to the strip can be measured.

The invention also aims at providing an ergometer that has a relatively simple and cost-effective structure.

This object is achieved according to the invention in that prior to the signal processing means the electric signals can be added together by means of an adding element.

The value to be determined by the signal processing means from the added-together electric signals is representative of the accelerations carried out by an individual such as a human being and is thus representative of the efforts made by the individual. With the aid of the ergometer according to the invention a value that is representative of the efforts can thus be determined in a simple manner.

An embodiment of the ergometer according to the invention is characterized in that the ergometer comprises a database in which the value is correlated to an energy value such as, for example, a nutritional value.

Such an embodiment is advantageous in that the value that is representative of the efforts of an individual can be coupled to an energy value such as, for example, a nutritional value to determine, for example, the number of kilojoules a certain effort has cost.

A further embodiment of the ergometer according to the invention is characterized in that the ergometer comprises a memory in which the energy values can be stored over a certain period of time.

In this way it is possible to compare values that are representative of individual efforts over time.

Another embodiment of the ergometer according to the invention is characterized in that the ergometer comprises a screen on which the instantaneous efforts and/or average effort over a certain period can be displayed in energy values.

Such an embodiment is advantageous in that the user can directly read the instantaneous effort and/or average effort.

Yet another embodiment of the ergometer according to the invention is characterized in that the ergometer comprises a coupling to which a computer can be connected for transferring stored data from the ergometer to the computer.

In this way the data can be transferred to a computer in which more storage space is present. In addition, the data can be processed more extensively in the computer and it is possible to make graphs of the effort recorded over time by the ergometer.

These and other aspects of the invention are apparent from and will be elucidated with reference to the embodiments described hereinafter.

In the drawings:

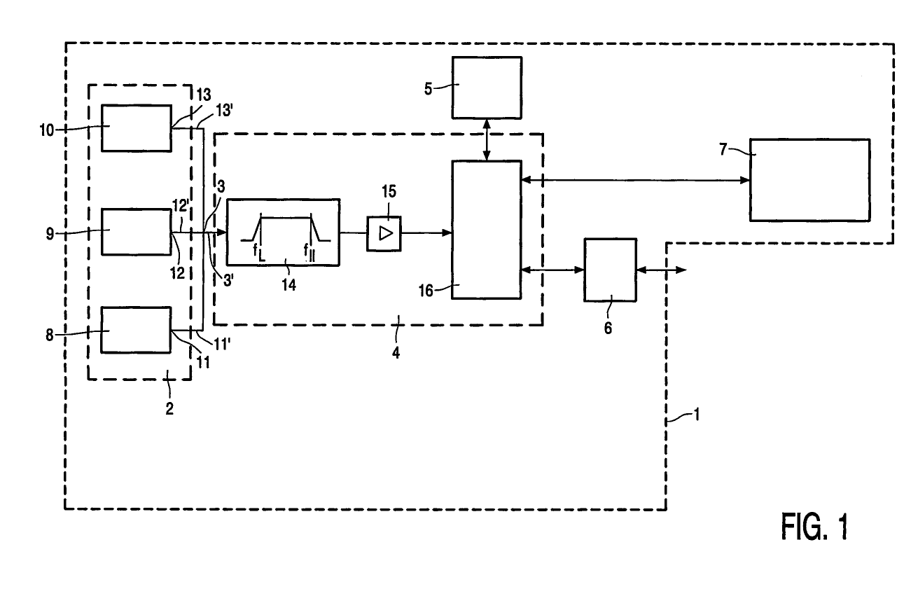

FIG. 1 shows a schematic view of a device according to the invention,

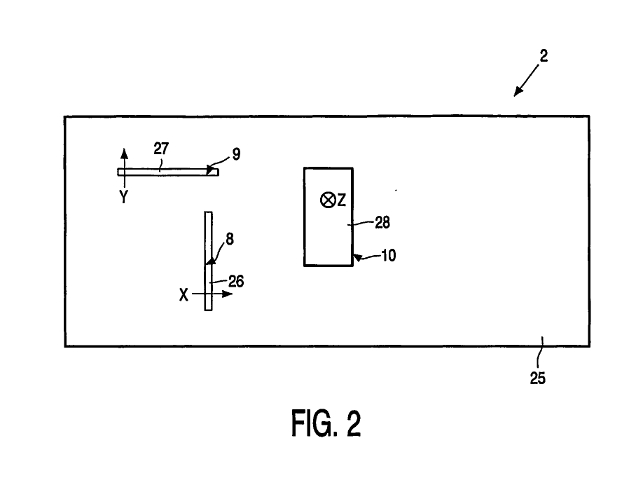

FIG. 2 shows a plan view of a sensor system of the device according to the invention shown in FIG. 1.

Like elements in the Figures have like references.

FIG. 1 shows a device according to the invention comprising a sensor system 2 which is connected to an adding element 3, signal processing means 4 connected to the adding element 3 and a memory 5 connected to the signal processing means 4, connection 6 and display means 7.

The sensor system 2 comprises three sensors 8, 9, 10 which have each an output 11, 12, 13. The outputs 11, 12, 13 of the sensors 8, 9, 10 are connected to the adding element 3 via parallel arranged electroconductive connections 11′, 12′, 13′, which adding element 3 is connected to the signal processing means 4 via a single electrical connection 3′. The signal processing means 4 comprise a filter arrangement 14, an amplifier 15 and a processor 16.

By means of the processor 16 a value is determined and possibly stored in a memory 19. A computer (not shown) is connected, for example serially connected, to the connection 6. The display means 7 such as a screen can display the values determined by means of the processor 16.

FIG. 2 shows the sensor system 2 of the device 1 according to the present invention. The sensor system 2 comprises a frame on which three sensors working independently 8, 9, 10 are installed.

The sensors 8, 9, 10 each comprise a piezoelectric strip 26, 27, 28 embedded in recesses in the frame 25, the strips 26, 27, 28 being retained at one end by means of a supporting agent (not shown). The strips 26, 27 extend through the frame 25, the strip 26 being oriented such that the acceleration in the X direction is recorded by means of the strip 26 and strip 27 being oriented such that the acceleration in the Y direction is recorded by means of the strip 27. The strip 28 is embedded in parallel with the frame 25 in the recess so that the acceleration in the Z direction is recorded by means of the strip 28.

The device, for example, attached to a belt is carried by a person. If this person walks or runs, forces will be exerted on the strips 26, 27, 28 as a result of the attendant accelerations, which forces lead to the strips 26, 27, 28 being bent. The bending will generate an electrical load which is proportional to the acceleration. The generated currents I₈, I₉, I₁₀ are transferred to the outputs 11, 12, 13 of the sensors 8, 9, 10.

The currents I₈, I₉, I₁₀ generated by means of the sensors 8, 9, 10 are representative of the accelerations in the X, Y, Z directions. The currents I₈, I₉, I₁₀ of the sensors 8, 9, 10 are led to an adding element 3 through the outputs 11, 12 and 13 and the connections 11′, 12′, 13′. In the adding element 3 the currents I₈, I₉, I₁₀ are added together, which produces a total current: Iₜₒₜ=I₈+I₉+I₁₀.

After the adding element 3 the total current Iₜₒₜ is processed to a value that can be processed by the processor 16. The value can be converted in the processor 16 into a magnitude that indicates the degree of effort. The values are correlated with energy values such as, for example, nutritional values. This is possible by means of a database stored in the processor 16 in which base each value has an associated energy value.

By means of the memory 19 it is, for example, possible to save the results for a certain period of time. The results can also be written in a computer via connection 6 with which computer the data produced by the device 1 can be processed still further.

The sensor system 2 may also have two sensors for recording, for example, only the acceleration in the X and Y directions.

It is alternatively possible to determine, prior to the adding element 3, the absolute value of each current coming from a sensor 8, 9, 10.

1. A device for determining a value that is representative of accelerations in at least two directions perpendicular to each other, the device comprising a sensor system with which the acceleration in each of the mutually perpendicular directions can be converted into an electric signal while the value can be determined from the electric signals by signal processing means, characterized in that prior to the signal processing means the electric signals can be added together by means of an adding element.

2. A device as claimed in claim 1, characterized in that in the adding element the connections conducting the electric signals are arranged in parallel.

3. A device as claimed in claim 1, characterized in that a sensor system comprises at least a sensor which comprises a flexible strip made of piezoelectric material.

4. A device as claimed in claim 1, characterized in that the signal processing means comprise a signal amplifier, a bandpass filter and a processor.

5. An ergometer for measuring a value that is representative of a physical effort of an individual, the ergometer comprising a device that includes a sensor system with which the acceleration in each of the directions extending perpendicularly to each other can be converted into electric signals, while the value can be determined from the electric signals by signal processing means, characterized in that prior to the signal processing means the electric signals can be added together by means of an adding element.

6. An ergometer as claimed in claim 5, characterized in that in the adding element the connections conducting the electric signals are arranged in parallel.

7. Ergometer as claimed in claim 5, characterized in that the ergometer comprises a database in which the value is correlated to an energy value such as, for example a nutritional value.

8. Ergometer as claimed in claim 7, characterized in that the ergometer comprises a memory in which energy values can be stored over a certain period of time.

9. Ergometer as claimed in claim 7, characterized in that the ergometer comprises a screen on which the instantaneous effort and/or average effort can be displayed in energy values over a certain period.

10. An ergometer as claimed in claim 5, characterized in that the ergometer comprises a coupling to which a computer can be connected, for transferring stored data from the ergometer to the computer.

11. An ergometer as claimed in claim 5, characterized in that the sensor system comprises at least a sensor that includes a flexible strip made of piezoelectric material.

12. An ergometer as claimed in claim 5, characterized in that the signal processing means comprise a signal amplifier, a bandpass filter and a processor.
